(19) United States
(12) Patent Application Publication
(10) Pub. No.: US 2023/0104349 A1
(43) Pub. Date: Apr. 6, 2023

(54) Halogenated Tetrasilyl Boranates

(71) Applicant: WACKER CHEMIE AG, Munich (DE)

(72) Inventors: Elke FRITZ-LANGHALS, Ottobrunn (DE); Sebastian BOCHMANN, Zschorlau OT Albernau (DE); Lars RUPPEL, Dresden-Langebrück (DE)

(73) Assignee: Wacker Chemie AG, Munich (DE)

(21) Appl. No.: 17/802,859

(22) PCT Filed: Feb. 26, 2020
(86) PCT No.: PCT/EP2020/054972

§ 371 Date: Aug. 26, 2022

(51) Int. Cl. B01J 27/06; C01B 35/06; B01J 31/02; B01J 37/04; C07F 7/16; C07F 5/02

(52) U.S. Cl. CPC B01J 27/06; C01B 35/06; B01J 31/0275; B01J 37/04; C07F 7/16; C07F 5/027; B01J 2531/002; B01J 2231/005

(57) ABSTRACT

The invention relates to halogenated tetrasilylboranates of the general formula

M^{z+}[B(SiRₘXₙ)₄⁻]_{z} (I),

where the radicals and indices have the meanings indicated in claim 1, with the proviso that m+n=3,
processes for the production thereof and also the use.

The invention relates to halogenated tetrasilylboranates, processes for the production thereof and also the use.

Tetrasilylboranates are already known. Mention may be made on this subject of, for example, the publication by Nöth et al. in Chem. Ber. 1982, 115, 934, in which the synthesis of Li⁺B(SiCH₃)₄⁻ by reaction of trimethoxyborane with trimethylsilyllithium under metal-organic conditions is described.

Compounds having a high acid strength are of great interest for industrial applications. They are frequently used catalytically and are therefore particularly valuable compounds. Furthermore, the halogenated tetrasilylboranates are weakly coordinating and stabilizing anions for organic cations which have great industrial importance as catalysts. Halogenated tetrasilylboranates are, in particular with the cation Ph₃C⁺, industrially important since they can easily be converted into catalytically active compounds; they are industrially important catalyst precursors.

Protic acid compounds are compounds which are able to release protons. The more weakly the proton is bound to the anion in the protic acid compound, the more easily it can be transferred to a substrate and the greater is its acid strength. High acid strengths are therefore possessed by, for example, tetrafluoroboric acid (H⁺BF₄⁻), perchloric acid (H⁺ClO₄⁻), trifluoromethanesulfonic acid (CF₃SO₃H) and hexafluoroantimonic acid (H⁺SbF₆⁻). These acids are also referred to as superacids since they have a very high acid strength. However, disadvantages of these acids are the difficulty of producing them, the difficulty of handling them because of their highly corrosive nature and their decomposability. Tetrafluoroboric acid is only stable in water or water-like solvents and can only be produced in solution. This also applies to perchloric acid. When the water content is reduced in the case of perchloric acid, there is a risk of explosions, and perchloric acid also has an oxidizing action, which represents a further disadvantage. Trifluoromethanesulfonic acid is produced by electrochemical fluorination of methanesulfonyl chloride, and hexafluoroantimonic acid is produced by reaction of anhydrous hydrogen fluoride with SbF₅. These processes can only be carried out in specific plants. These properties of the known very strong acids therefore make the industrial use thereof considerably more difficult.

Compounds having a high acid strength are suitable as catalysts which catalyze the conversion of Si—H groups into the corresponding halogen groups. Thus, for example, DE-A 102007030948 describes a process for converting Si—H into Si—Cl, in which tetrabutylphosphonium chloride is used as catalyst and gaseous HCl is used as chlorinating agent. A disadvantage here is that gaseous HCl is comparatively difficult to handle. DE-A 4240717 describes a further process for converting Si—H into Si—Cl with the aid of allyl chloride and palladium catalysts or platinum catalysts. However, noble metal compounds are costly and therefore have to be recycled, which leads to high process costs.

A process in which the conversion of Si—H into Si—Cl by means of dichloromethane is carried out by irradiation in the presence of 1 mol % of Eosin Y in a specific irradiation apparatus is described in Angew. Chem 2019, 131, 12710. However, this process is technically very complicated, and in addition the dye Eosin is undesirable in the industrial products.

It is therefore an object of the present invention to find, inter alia, compounds which do not have the abovementioned disadvantages.

The present invention accordingly provides halogenated tetrasilylboranates of the general formula

M^{z+}[B(SiRₘXₙ)₄⁻]_{z} (I),

where
M^{z+} is an inorganic or organic cation where z is 1 or 2, preferably 1,
R is identical or different on each occurrence and is a hydrogen atom or hydrocarbon radical having from 1 to 3 carbon atoms,
X is identical or different on each occurrence and is a halogen atom,
m is 0, 1 or 2, preferably 0 or 1, particularly preferably 0, and
n is 1, 2 or 3, preferably 2 or 3, particularly preferably 3,
with the proviso that m+n=3.

The radical X is preferably F, Cl or Br, particularly preferably F or Cl, in particular Cl. The radical R is preferably a hydrogen atom or the methyl radical.

Examples of the cation M^{z+} are H⁺, cations of the alkali metals and alkaline earth metals, cationic nitrogen compounds, phosphonium cations and carbocations.

The cations M^{z+} are preferably H⁺, Li⁺, Na⁺, K⁺, Cs⁺, Mg⁺, Ca²⁺, Ba²⁺, nitrogen compounds of the formulae NR⁴₄⁺ and ═NR⁵₂⁺, where R⁴ and R⁵ can be identical or different in each case and are each a hydrogen atom or a C1-C20 alkyl, aryl or aralkyl radical which can in each case be interrupted by heteroatoms, where two or more of the C1-C20 radicals can form one or more rings which can optionally be (hetero)aromatic, phosphonium cations PR⁶₄⁺, where the radicals R⁶ can be identical or different and are each a halogen atom, in particular chlorine atom, or a C1-C20 alkyl, aryl or aralkyl radical, or carbocations of the general formula R⁷₃C⁺, where the radicals R⁷ can be identical or different and are each an aryl radical which may optionally be substituted.

The cations M^{z+} are particularly preferably H⁺ or Ph₃C⁺, in particular H⁺, where Ph is a phenyl radical.

Although not shown in formula (I), the cation M^{z+}, in particular the proton H⁺, in the compound of the invention can also be complexed by oxygen-containing electron donors (D).

Oxygen-containing electron donors (D) are, for example, ethers or alcohols of the general formula (II)

R¹—O—R² (II),

where R¹ is a hydrocarbon radical having from 1 to 20 carbon atoms and R² is a hydrogen atom or a hydrocarbon radical having from 1 to 20 carbon atoms.

Examples of radicals R¹ are alkyl radicals such as the methyl, ethyl, n-propyl, isopropyl, n-butyl, isobutyl, tert-butyl, n-pentyl, isopentyl, neopentyl, tert-pentyl radical, hexyl radicals such as the n-hexyl radical, heptyl radicals such as the n-heptyl radical, octyl radicals such as the n-octyl radical and isooctyl radicals such as the 2,2,4-trimethylpentyl radical, nonyl radicals such as the n-nonyl radical, decyl radicals such as the n-decyl radical, dodecyl radicals such as the n-dodecyl radical; alkenyl radicals such as the vinyl radical and the allyl radical; cycloalkyl radicals such as cyclopentyl, cyclohexyl, cycloheptyl radicals and methylcyclohexyl radicals; aryl radicals such as the phenyl radical and the naphthyl radical; alkaryl radicals such as o-, m-, p-tolyl radicals, xylyl radicals and ethylphenyl radicals; and also aralkyl radicals such as the benzyl radical, the α-phenylethyl radical and the β-phenylethyl radical.

Examples of radicals R² are the examples indicated for radicals R¹ and also the hydrogen atom.

The radicals R¹ and R² are, independently of one another, preferably alkyl radicals having from 1 to 6 carbon atoms, particularly preferably methyl, ethyl, n-propyl or isopropyl radicals.

The electron donors (D) are preferably diethyl ether, diisopropyl ether, di-n-propyl ether, dibenzyl ether, methoxybenzene, methanol, ethanol, n-propanol and n-butanol.

Examples of the tetrasilylboranates of the formula (I) according to the invention are H⁺B(SiCl₃)₄⁻, H⁺B(SiHCl₂)(SiCl₃)₃⁻, H⁺B(SiHCl₂)₂(SiCl₃)₂⁻, H⁺B(SiHCl₂)₃(SiCl₃)⁻, H⁺B(SiHCl₂)₄⁻, Li⁺B(SiCl₃)₄⁻, Nn₄⁺B(SiCl₃)₄⁻, Et₃NH⁺B(SiCl₃)₄⁻, Et₂NH₂⁺B(SiCl₃)₄⁻, C₅H₅NH⁺B(SiCl₃)₄⁻, imidazolium ⁺B(SiCl₃)₄⁻, Ph₄P⁺B(SiCl₃)₄⁻, Bu₄P⁺B(SiCl₃)₄⁻, Me₄P⁺B(SiCl₃)₄⁻ and Ph₃C⁺B(SiCl₃)₄⁻, with preference being given to H⁺B(SiCl₃)₄⁻, H⁺B(SiHCl₂)(SiCl₃)₃⁻ or Ph₃C⁺B(SiCl₃)₄⁻, particularly preferably H⁺B(SiCl₃)₄⁻ or Ph₃C⁺B(SiCl₃)₄⁻, in particular H⁺B(SiCl₃)₄⁻, where Me is the methyl radical, Et is the ethyl radical, Bu is the butyl radical and Ph is the phenyl radical.

The compounds H⁺B(SiCl₃)₄⁻ and Ph₃C⁺B(SiCl₃)₄⁻ according to the invention surprisingly display a high thermal stability. H⁺B(SiCl₃)₄⁻ melts without decomposition at 187° C. and can be cooled below the melting point and melted again to above 200° C. a number of times without decomposition. Decomposition is observed only at significantly higher temperatures of more than 200° C.

The tetrasilylboranates of the invention can be produced by processes known per se, preferably by reaction of boron trihalides with halosilanes.

The present invention therefore further provides a process for producing the tetrasilylboranates according to the invention by reaction of boron trihalides with at least two different halosilanes bearing Si-bonded hydrogen, wherein the boranate obtained in this way is reacted with a proton acceptor (B) in an optionally performed further step.

The proton acceptors (B) which may optionally be used according to the invention are preferably M′^{z+}(OH)_{z} where M′ represents cations of the alkali metals with z=1 and alkaline earth metals with z=2, ammonium hydroxide of the formula NR^{4′}₄⁺OH⁻, immonium hydroxide of the formula=NR^{5′}₂⁺OH⁻, where R^{4′} and R^{5′} can in each case be identical or different and are each C1-C20 alkyl, aryl or aralkyl radicals which may be interrupted by heteroatoms, where two or more of the C1-C20 radicals can form one or more rings which may optionally be (hetero)aromatic, phosphonium hydroxides of the formula PR^{6′}₄⁺OH⁻, where R^{6′} can be identical or different on each occurrence and has the meaning C1-C20 alkyl, aryl or aralkyl radical, carbinols of the formula R⁷₃COH where R⁷ can have the meaning indicated above or be a nitrogen base, preferably R⁴₃N or ═NR⁵, where R⁴ and R⁵ have the meanings indicated above.

In the process of the invention, boron trihalides BX₃ are preferably reacted with silanes (S1) of the formula HSiRₘXₙ and silanes (S2) of the formula H₂SiR_{m′}X_{n′}, where the radicals R and X can in each case be identical or different and have the abovementioned meanings, m and n have the abovementioned meanings, m′ is 0 or 1, preferably 0, and n′ is 1 or 2, preferably 2, where m+n=3 and m′+n′=2.

The silanes (51) used according to the invention are preferably silanes of the formula HSiX₃ where X has the abovementioned meaning, with particular preference being given to trichlorosilane.

The silanes (S2) used according to the invention are preferably silanes of the formula H₂SiX₂ where X has the abovementioned meaning, with particular preference being given to dichlorosilane.

In the process of the invention, the molar ratio of the boron halides BX₃ to the molar sum of the silanes (Si) and (S2) is preferably at least 1:0.1 and not more than 1:10¹⁰, particularly preferably at least 1:1 and not more than 1:10⁸, in particular at least 1:10 and not more than 1:10⁶.

In the process of the invention, the molar ratio of the silanes (Si) to the silanes (S2) is preferably in the range from 10⁸:1 to 1:10⁶, particularly preferably from 10⁵:1 to 1:10⁴, in particular from 10²:1 to 1:10², very particularly preferably from 20:1 to 1:20.

The reaction according to the invention is preferably carried out at temperatures in the range from −20 to +400° C., particularly preferably from 0° C. to +200° C., in particular from +20° C. to +100° C.

The reaction according to the invention is preferably carried out at pressures of from 10 to 100 000 hPa, particularly preferably from 100 hPa to 10 000 hPa.

The reaction can also be carried out in the presence of metallic surfaces, preferably transition metal surfaces, particularly preferably iron, chromium, nickel, manganese or alloys thereof, in particular stainless steels.

The reaction according to the invention is preferably carried out under protective gas, for example nitrogen and argon. It can be carried out with or without addition of solvent, with the reaction without solvent being preferred. If the reaction is carried out using solvents, preference is to be given to saturated hydrocarbons, aromatic hydrocarbons or ethers, preferably in proportions of from 1% by weight to 90% by weight, in each case based on the total weight of the reaction mixture.

The protic acid halogenated tetrasilylboranate produced according to the invention precipitates from the reaction mixture and can therefore be separated off very easily. The reaction according to the invention preferably does not give rise to any waste products. Excess reagents can be reused.

The acids obtained according to the invention can, if desired, be reacted with proton acceptors (B) in order to obtain compounds of the formula (I) in which M^{z+} is not H⁺. This reaction is preferably carried out at ambient temperature and ambient pressure, preferably with stirring, in the presence of one or more inert solvents, for example ethers, chlorinated hydrocarbons or dipolar aprotic solvents such as nitriles, amides or dimethyl sulfoxide.

The process of the invention for producing the tetrasilylboranates of the formula (I) can be carried out continuously, discontinuously or semicontinuously.

The compounds of the invention can be used for all purposes for which boranates have hitherto also been used. The inventive compounds of the formula (I) where M^{z+} is hydrogen can also be used for all purposes for which strong acids are required. For example, salts of tritylium cations, Ph₃C⁺, have hitherto been produced by reacting Ph₃COH with strong acids such as HBF₄, HPF₆, HClO₄, HSO₃F and methanesulfonic acid. Surprisingly, Ph₃COH can very easily be converted into the compound Ph₃C⁺B(SiCl₃)₄⁻ in an analogous way by reaction with the inventive compound H⁺B(SiCl₃)₄⁻ with elimination of water.

The inventive compounds of the formula (I) where X is Cl, in particular the compound H⁺B(SiCl₃)₄⁻, are preferably catalysts suitable for industrial use which catalyze the conversion of silanes and siloxanes having Si—H groups into the corresponding chlorosilanes or chlorosiloxanes in the presence of chlorinated hydrocarbons.

The invention therefore further provides a process for converting compounds (H) bearing Si-bonded hydrogen into the corresponding compounds bearing Si-bonded halogen atoms by reaction with halogenated hydrocarbons (K) in the presence of compounds of the general formula (I) where X is Cl and M^{z+} is H⁺ as catalyst.

The conversion of Si—H into Si-halogen groups is industrially important since residual contents of hydridosiloxanes in silicone materials can lead to formation of hydrogen gas during storage.

The compounds (H) bearing Si-bonded hydrogen which are used according to the invention can be all previously known organosilicon compounds having Si-bonded hydrogen, preferably compounds composed of units of the formula (III)

R³ₐY_{b}H_{c}SiO_{(4-a-b-c)/2} (III),

where
R³ can be identical or different on each occurrence and is a monovalent, optionally substituted hydrocarbon radical which can be interrupted by heteroatoms,
Y can be identical or different on each occurrence and is a halogen atom or organyloxy radical,
a is 0, 1, 2 or 3,
b is 0, 1, 2 or 3 and
c is 0, 1 or 2, preferably 0 or 1,
with the proviso that c≠0, in at least one unit and the sum a+b+c is ≤4.

The organosilicon compounds (H) used according to the invention can be either silanes, i.e. compounds of the formula (III) with a+b+c=4, or siloxanes, i.e. compounds made up of units of the formula (III) where a+b+c≤3. The organosilicon compounds used according to the invention are preferably silanes.

Examples of radicals R³ are the examples given for radicals R¹, with the radicals R³ also being able to be substituted by halogen radicals.

Radicals R³ are preferably hydrocarbon radicals having from 1 to 12 carbon atoms which can optionally be monochlorinated or polychlorinated, particularly preferably C1-C6 alkyl radicals, phenyl radicals, vinyl radicals or allyl radicals which may optionally be monochlorinated or polychlorinated, in particular the methyl, ethyl, vinyl, allyl, chloromethyl, 3-chloropropyl or phenyl radical.

The radical Y is preferably a halogen atom, particularly preferably a chlorine atom.

Examples of compounds (H) used according to the invention are methyldichlorosilane, dimethylchlorosilane, trichlorosilane, ethyldichlorosilane, methylethylchlorosilane, trimethylsilane, phenylmethylchlorosilane, vinylmethylchlorosilane, divinylchlorosilane, allylmethylchlorosilane and diphenylchlorosilane.

The halogenated hydrocarbons (K) used according to the invention can be any, previously known hydrocarbons in which one or more hydrogen atoms have been replaced by halogen atoms, with compounds (K) being able to be linear, branched, cyclic, saturated, aliphatically unsaturated or aromatic.

Examples of the halogenated hydrocarbons (K) used according to the invention are dichloromethane, chloromethane, chloroform, 1,2-dichloroethane, 2-chloropropane, chlorobenzene, o-dichlorobenzene, allyl chloride or benzyl chloride.

The halogenated hydrocarbons (K) used according to the invention are preferably hydrocarbons having from 1 to 50 carbon atoms in which one or more hydrogen atoms have been replaced by halogen atoms, in particular chlorine atoms, particularly preferably chlorinated hydrocarbons having from 1 to 20 carbon atoms, in particular chloromethane, dichloromethane, chloroethane, 1-chloropropane, 2-chloropropane, 1,3-dichloropropene, 1,2-dichloroethane, 1,1,1-trichloroethane, allyl chloride, benzyl chloride, chlorobenzene or ortho-dichlorobenzene.

In the process of the invention, the molar ratio of Si—H groups in the organosilicon compounds (H) to C—Cl groups in the compounds (K) is preferably at least 100:1 and not more than 1:10⁶, particularly preferably at least 10:1 and not more than 1:1000, in particular at least 2:1 and not more than 1:100.

The reaction according to the invention is preferably carried out under protective gas, for example nitrogen and argon.

In the process of the invention for converting compounds (H) bearing Si-bonded hydrogen into the corresponding compounds bearing Si-bonded halogen atoms, inert solvents (L) can be additionally used, with preference being given to aliphatic or aromatic hydrocarbons having from 3 to 50 carbon atoms. If solvents (L) are used in the process of the invention, they are preferably used in amounts of from 1% by weight to 99% by weight, particularly preferably from 10% by weight to 90% by weight, in each case based on the reaction mixture. The use of solvents (L) is not preferred.

The process of the invention is preferably carried out at pressures in the range from 500 hPa to 50 000 hPa, particularly preferably at ambient pressure, i.e. a pressure in the range from 900 to 1100 hPa.

The reaction according to the invention is preferably carried out at temperatures in the range from −20° C. to +200° C., particularly preferably from 0° C. to +100° C.

The process of the invention for converting compounds (H) bearing Si-bonded hydrogen into the corresponding compounds bearing Si-bonded halogen atoms can be carried out continuously, discontinuously or semicontinuously, with a continuous reaction being preferred.

The compounds of the invention, in particular the protic acid halogenated tetrasilylboranates and the tritylium salts thereof, have the advantage that they have a high stability and owing to their nonvolatility can be handled in a very simple manner.

Organic cations are stabilized very well by the anion according to the invention and can therefore be used advantageously in industrial processes. In particular, their high stability is advantageous for catalytic processes since consumption of additional amounts is avoided thereby.

The process of the invention for producing the compounds of the formula (I) is simple to carry out and it is possible to use industrially available, inexpensive starting materials such as chlorosilanes and boron trichloride.

The process of the invention also has the advantage that no waste products which have to be recycled or disposed of are formed.

The use according to the invention of the compounds of the formula (I) has the advantage that Si-bonded hydrogen can be converted into Si-bonded halogen atoms in a simple and efficient way.

The process of the invention for converting Si-bonded hydrogen into Si-bonded halogen can advantageously also be used for converting halogen-substituted hydrocarbons into halogen-free hydrocarbons. This is likewise of interest in industry since halogenated hydrocarbons are frequently toxic compounds, the disposal of which is complicated. The halosilane obtained can be eliminated in a simple manner by hydrolysis in water.

In the following examples, all parts and percentages are, unless indicated otherwise, by weight. Unless indicated otherwise, the following examples are carried out at a pressure of the surrounding atmosphere, i.e. at about 1000 hPa, and at room temperature, i.e. about 20° C. or a temperature which is established on combining the reactants at room temperature without additional heating or cooling.

### EXAMPLE 1

### Synthesis and Characterization of H⁺B(SiCl₃)₄

50 g of trichlorosilane and 2 g of dichlorosilane are placed under a nitrogen atmosphere at 0° C. in a steel autoclave. 20 mg of boron trichloride are introduced while stirring. The autoclave is closed and allowed to stand for 20 hours at 70° C. with pressure regulation at a gauge pressure of about 2 bar. The reaction mixture is devolatilized at atmospheric pressure at a liquid-phase temperature of up to about 30° C. The autoclave is then closed again and operated under a nitrogen atmosphere with pressure regulation at a gauge pressure of 1 bar for 100 hours at 55° C.

Finally, evaporation of the resulting reaction solution gives 40 mg of a crystalline residue of H⁺B(SiCl₃)₄⁻, which is characterized as follows: melting point 187° C.; ²⁹Si-NMR(CD₂Cl₂, 99.4 MHz): □□□=19.8 ppm (q, ¹J_{Si,B}=89.0 Hz), ¹¹B-NMR (CD₂Cl₂, 160 MHz): □□=−26.84 ppm.

### EXAMPLE 2

### Synthesis of H⁺B(SiCl₃)₄

A mixture of 100 g of trichlorosilane with 5 g of dichlorosilane and 55 mg of boron trichloride is allowed to stand at 70° C. while stirring and under a nitrogen atmosphere in a steel autoclave with pressure regulation at a gauge pressure of 2 bar for 24 hours. Subsequent devolatilization at about 30° C. is followed by renewed reaction in the closed steel autoclave at a gauge pressure of 1 bar and 55° C. for 120 hours. Evaporation of the reaction solution gives 140 mg of H+B(SiCl₃)₄⁻.

### EXAMPLE 3

### Production of Ph₃C⁺B(SiCl₃)₄⁻

Under argon, 101 mg (0.18 mmol) of H⁺B(SiCl₃)₄⁻ are dissolved in 3.36 g of d₆-benzene and, while stirring, a solution of 46.8 mg (0.18 mmol) of triphenylmethanol in 823 mg of d₆-benzene is added dropwise. The reaction solution acquires a dark-yellow color and the product precipitates as orange-colored solid which settles at the bottom. The supernatant solution is decanted off and the solid (product) is washed with a little d₆-benzene and dried at room temperature under reduced pressure. The yield is 180 mg (90%).

¹H-NMR (CD₂Cl₂, 500 MHz): □□□=7.70 (mc, 6 aromat. H), 7.93 (mc, 6 aromat. H), 8.31 (mc, 3 aromat. H); ¹³C-NMR (CD₂Cl₂, 126 MHz): □□=130.7, 139.9, 142.8, 143.7 ppm; ²⁹Si-NMR(CD₂Cl₂, 99.4 MHz): □□□=21.58 ppm (q, ¹J_{Si,B}=89.0 Hz), ¹¹B-NMR (CD₂Cl₂, 160 MHz): □=−30.74 ppm.

### EXAMPLE 4

### Production of methyltrichlorosilane

A solution of 102 mg (0.90 mmol) of methyldichlorosilane in 770 mg of dichloromethane is admixed while shaking with a solution of 0.29 mg (0.53 μmol, 0.059 mol %) of H⁺B(SiCl₃)₄⁻ in 43 mg of dichloromethane. The reaction mixture is allowed to stand at 23° C. in the closed vessel and the formation of methyltrichlorosilane is examined by NMR spectroscopy: 13 mol % (45 min), 42 mol % (3 hours), 99 mol % conversion (20 hours). Chloromethane and methane are additionally formed.

¹H-NMR (CD₂Cl₂, 500 MHz): δ=1.17 (s, CH₃); ²⁹Si-NMR (CD₂Cl₂, 500 MHz): δ=12.72 ppm.

### EXAMPLE 5

### Production of methyltrichlorosilane

A solution of 102 mg (0.90 mmol) of methyldichlorosilane in 800 mg of d6-benzene is admixed while shaking with a solution of 0.44 mg (0.81 μmol, 0.09 mol %) of H⁺B(SiCl₃)₄⁻ in 49 mg of d6-benzene. Chloromethane is passed into the solution and the amount thereof is determined by ¹H-NMR spectroscopy: 67 mg (1.3 mmol). The reaction mixture is allowed to stand in the closed vessel at 23° C.; methyltrichlorosilane and methane are formed. Conversion into methyltrichlorosilane: 2 mol % (40 minutes), 10 mol % (1.6 hours), 39 mol % conversion (4.6 hours), 52 mol % conversion (30 hours), 100 mol % conversion (3 days).

¹H-NMR (d6-benzene, 500 MHz): δ=1.17 (s, CH₃); ²⁹Si-NMR (CD₂Cl₂, 500 MHz): δ=12.72 ppm.

¹H-NMR (d6-benzene, 500 MHz) of the product methane: δ=0.22.

### EXAMPLE 6

### Production of dimethyldichlorosilane

A solution of 0.50 mg (0.91 μmol) of H⁺B(SiCl₃)₄⁻ in 620 mg of dichloromethane is admixed while shaking with a mixture of 155 mg (2.02 mmol) of allyl chloride and 130 mg (1.38 mmol) of dimethylchlorosilane. The mixture heats up briefly to 37° C. and then cools down to room temperature again. GC analysis indicates complete conversion and 80% by weight of dimethyldichlorosilane. In addition, propene is formed.

### EXAMPLE 7

### Production of chloropentamethyldisiloxane

3.5 mg (6.6 μmol) of H⁺B(SiCl₃)₄⁻, 152 mg (1.99 mmol) of allyl chloride and 196 mg (1.32 mmol) of pentamethyldisiloxane are mixed. GC analysis after a reaction time of 20 hours indicates 62% by weight of chloropentamethyldisiloxane. In addition, propene is formed.

### EXAMPLE 8

### Production of di-tert-butyldichlorosilane

154 mg (2.01 mmol) of allyl chloride and 234 mg (1.32 mmol) of di-tert-butylchlorosilane are mixed and admixed with a solution of 3.6 mg (6.5 mmol) of H⁺B(SiCl₃)₄⁻ in 300 mg of dichloromethane. An exothermic reaction with formation of propene takes place, leading to formation of di-tert-butyldichlorosilane.

Yield (GC): 83% by weight.

¹H-NMR (CD₂Cl₂, 500 MHz): δ=1.22 (s, tert-butyl).

1-8. (canceled)

9. A halogenated tetrasilylboranate, comprising:
wherein the halogenated tetrasilylboranate has the general formula
M^{z+}[B(SiRₘXₙ)₄⁻]_{z} (I),
wherein M^{z+} is an inorganic or organic cation;
wherein z is 1 or 2, preferably 1;
wherein R is identical or different on each occurrence and is a hydrogen atom or hydrocarbon radical having from 1 to 3 carbon atoms;
wherein X is identical or different on each occurrence and is a halogen atom;
wherein m is 0, 1 or 2;
wherein n is 1, 2 or 3; and
wherein m+n=3.

10. The halogenated tetrasilylboranate of claim 9, wherein M^{z+} is H⁺ or Ph₃C⁺.

11. The halogenated tetrasilylboranate of claim 9, wherein X is F or Cl.

12. The halogenated tetrasilylboranate of claim 9, wherein it is H⁺B(SiCl₃)₄⁻, H⁺B(SiHCl₂)(SiCl₃)₃⁻ or Ph₃C⁺B(SiCl₃)₄⁻.

13. A process for producing a tetrasilylboranates, comprising:
reacting boron trihalides with at least two different halosilanes bearing Si-bonded hydrogen, and wherein the boranate obtained in this way is reacted with a proton acceptor (B) in an optionally performed further step.

14. The process of claim 13, wherein boron trihalides BX₃ are reacted with silanes (S1) of the formula HSiRₘXₙ and silanes (S2) of the formula H₂SiR_{m′}X_{n′};
wherein the radicals R in each case can be identical or different and is a hydrogen atom or hydrocarbon radical having from 1 to 3 carbon atoms;
wherein X in each case can be identical or different and is a halogen atom;
wherein m is 0, 1 or 2;
wherein n is 1, 2 or 3;
wherein m′ is 0 or 1;
wherein n′ is 1 or 2;
wherein m+n=3; and
wherein m′+n′=2.

15. A process for converting compounds (H) bearing Si-bonded hydrogen into the corresponding compounds bearing Si-bonded halogen atoms by a reaction with halogenated hydrocarbons (K) in the presence of compounds of the general formula (I) wherein X is Cl and M^{z+} is H⁺ as catalyst.

16. The process of claim 15, wherein the molar ratio of Si—H groups in the organosilicon compounds (H) to C—Cl groups in the compounds (K) is at least 100:1 and not more than 1:10⁶.
